(19) United States
(12) Patent Application Publication
(10) Pub. No.: US 2021/0100983 A1
(43) Pub. Date: Apr. 8, 2021

(54) INFUSION SITE PROTECTOR

(71) Applicant: Lindi Caywood, Fort Mill, SC (US)

(72) Inventor: Lindi Caywood, Fort Mill, SC (US)

(21) Appl. No.: 16/634,625

(22) PCT Filed: Jul. 17, 2018
(86) PCT No.: PCT/US18/42549

§ 371 Date: Jan. 28, 2020

Related U.S. Application Data
(60) Provisional application No. 62/545,826, filed on Aug. 15, 2017.

(51) Int. Cl. A61M 25/02; A61M 5/142

(52) U.S. Cl. CPC A61M 25/02; A61M 2025/0266; A61M 2025/0246; A61M 5/142

(57) ABSTRACT

Disclosed are devices and methods for providing comfort to an infusion patient as well as increased patient compliance due to increased comfort. The device includes a protective cover having a main body having an inner surface, an outer surface and a circumferential flange. The main body has a raised outer surface overlying a hollow cavity. The flange has one or more grooves adapted to provide clearance for one or more infusion set tubes to pass between the patient's skin and the cover.

The present disclosure relates to medical protective devices and shields, and more particularly to medical device accessories and methods for shielding and protecting infusion sites or needle/cannula injection sites from physical irritation, pulling, rubbing and bumping, thereby increasing patient comfort and compliance.

Presently, there is nothing available to protect infusion sites, especially long-term use sites, from irritation that is caused by clothing rubbing or being bumped or a patient simply laying on top of the site. When infusion sites are pulled, bumped or rubbed by clothing, the cannula or needle that pierces the skin moves, and the site can become irritated and sore. This is especially true of the long-term use micro infusion needles or soft sets that a patient may use for up to 3 days. Also, when pulled, bumped or rubbed, the infusion set can be pulled partially off the patient or it can even become infected, after the infusion set is loosened and the sterile seal is broken due to movement. Additionally, many patients stop using portable medication or infusion pumps because of site soreness and infections.

Disclosed herein are devices and methods related to embodiments of a medical device accessory that is designed to provide increased comfort to a patient as well as increased patient compliance due to increased comfort. The disclosed infusion site cover will lessen or prevent irritation and pain at the site by protecting the site from the rub of clothing or bumps of daily life. By decreasing the pain and discomfort at the infusion site, more people with medication pumps will continue using their pumps thereby increasing patient compliance of their medication or drugs therapeutic regime.

In one aspect the present disclosure provides a protective cover or protective device for an infusion site, comprising a main body having an inner surface, an outer surface and a circumferential flange, wherein the body has a raised outer surface, preferably a rounded or elongated dome shape, or a round donut shape, overlying a hollow cavity and the flange has one or more grooves adapted to provide clearance for one or more infusion set tubes to pass between the patient's skin and the cover, when the protective cover is installed over an infusion site.

The present disclosure also provides a protective cover or protective device for an incision site, comprising a main body having an upper surface, a bottom surface, and an exterior side surface defining a hollow; and, a through hole adapted to accommodate encircling an infusion set, passing through the upper and bottom surfaces; wherein the main body is formed of a resiliently a deformable plastic, gel or foam that returns to its original shape after being deformed.

In a preferred embodiment, wherein the main body has a smooth exterior surface, and an adhesive on the bottom surface to allow the protective cover to be adhered to the patient's skin. If desired, the adhesive comprises a pressure sensitive adhesive, and having a release layer over the adhesive. Also, in one embodiment, the main body has at least one vertical cut connecting the through hole to an outward exterior side surface of the main body.

The disclosed protective cover or device typically is manufactured of a sturdy plastic or other semi rigid material designed to fit over or around the infusion site of a medication pump or needle/cannula injection site. The material may be manufactured to a specified range of hardness or flexibility to enable the maximum protection and comfort. More specifically the material is manufactured in a Hardness range of Shore 00 30 to Shore 00 80, preferably a Hardness range of Shore 00 40 to 00 70, more preferably a Hardness range of Shore of 00 50 to Shore 00 60.

The disclosed protective cover or device may or may not contain air holes for ventilation. For example, in one embodiment the protective cover may be manufactured via injection molding of a relatively rigid plastic mesh that allows air flow to the site while the flange area is solid without holes to provide the most area for an application of adhesive or tape. Another embodiment of the disclosed invention may be manufactured of a rigid plastic that only has one large hole. While yet another embodiment may be a solid round donut shape with a convex profile, that is higher at the center, manufactured from a relatively soft gel like plastic.

Additionally, the disclosed protective cover or device may contain one or more notches or grooves in the flanged area of the protective cover that adheres to the patient's skin. The notches or grooves allow for the infusion tubing that fluidically connects the infusion site needle or cannula and the medication pump. The notches or grooves are necessary to prevent crimping or mashing of the infusion tubing. If the tubing were to crimp, kink or mash, flow of medication would stop or be severely impeded and reduced.

The flanged edge of the protective cover or device provides additional surface area for adhesion than a straight edge, and the flanged edge additionally provides structural rigidity. The flanged edge may or may not have padding adapted to be adhered to the flange area so that it is located between the patient's skin surface and the device as well on the surface in contact with the patient's skin. The padding provides greater comfort for the wearer. The size, geometric shape, and vertical clearance can be different in the disclosed invention to accommodate differences in various infusion set products, brands and makes, since various cannula housings of different manufacturers are often different in color, size, shape, height and materials.

An exemplary embodiment of the disclosed protective cover or device may include hypoallergenic tape or adhesive attached to the flange area to facilitate adhesion of the protective cover to the patient. Additionally, a gauze, foam or other hypoallergenic type of material may be added to provide padding to the protective cover to help lessen any discomfort. The padding is adapted to attach to the protective cover flange area so that in use it would be located between the protective cover and the patient's skin. The padding may serve as a dual purpose both for padding and as a spacer to provide the protective cover additional height if needed.

Further examples of embodiments of the disclosed protective cover or device may include precut tape or adhesive film that is shaped to fit the geometric shape of the protective cover, this may be in the form of a two sided adhesive tape that is cutout to fit the shape of the flange area, or another example is a one sided adhesive tape wherein the center is cut out to fit over the top raised area of the protective cover and attach to the top exterior side of the flange area with the tape extending outward from the flange, to adhere to the patient. The flange area also may be coated, at least in part, by a pressure sensitive adhesive which in turn is protected by a release cover.

In yet another embodiment the protective cover or device is attached to a cannula or infusion set needle inserting device in such a way that the protective cover is released from the inserting device and attached to the patient at the same time as the cannula, infusion set needle or soft set is installed or inserted into the patient. The protective cover is loaded onto the inserting device when the cannula, infusion set needle or soft set are loaded. The inserting devices are typically spring loaded and once loaded with the infusion set of choice, the medical provider, or the patient in the case of self-administration, presses the inserter against the patient's skin and pushes a button or trigger, the cannula or infusion site needle is quickly inserted penetrating through the patient's skin using a needle that is either left inserted in the patient or retracted from the soft cannula that is left in the patient. The infusion set hub is additionally adhered to the patient at the same time. Various changes of the inserter device would additionally allow the protective covers to be loaded and adhered to the patient at the same time as the cannula, infusion set needle and infusion set hub. Applying the protective cover at the same time would aid in keeping the sterile integrity of the area intact.

The details of one or more variations of the subject matter described herein are set forth in the accompanying drawings and the description below. Other features and advantages of the subject matter described herein will be apparent from the description, drawings and from the claims.

These and other aspects will now be described in detail with reference to the following drawings, wherein like numerals depict like parts, and wherein:

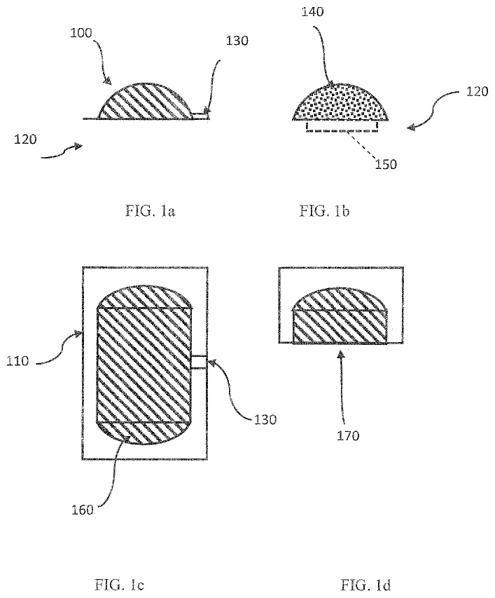

FIGS. 1a-1d show an embodiment of a medical device accessory, the protective cover configured to fit over an infusion set.

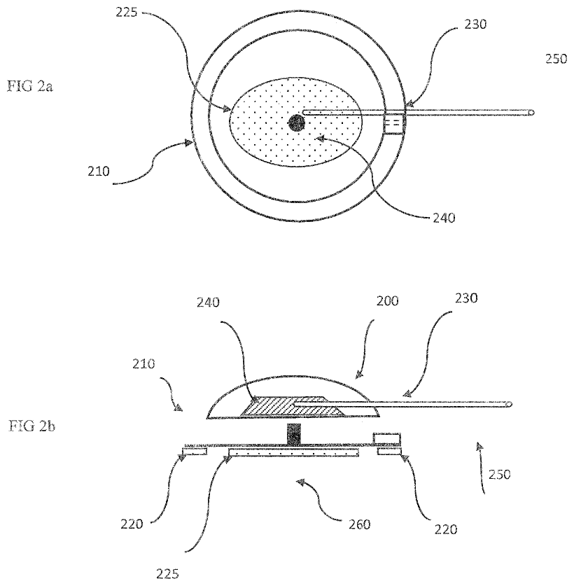

FIGS. 2a-2b show an embodiment of a rigid protective cover configured to fit over an infusion set that is adhered to a patient.

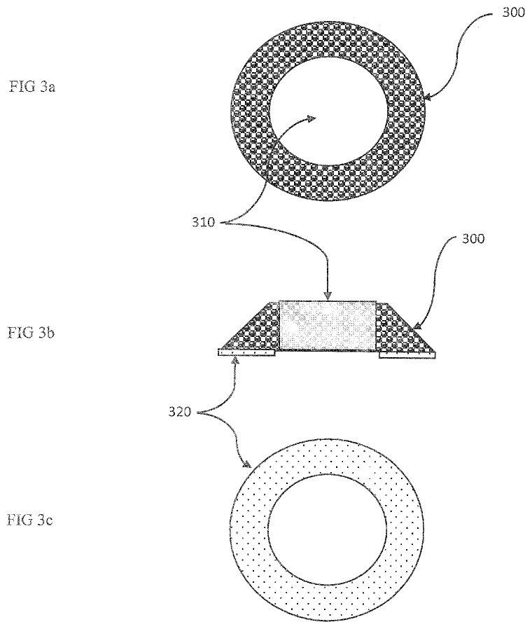

FIGS. 3a-3c show an embodiment of a soft plastic gel protective cover configured to fit over an infusion set.

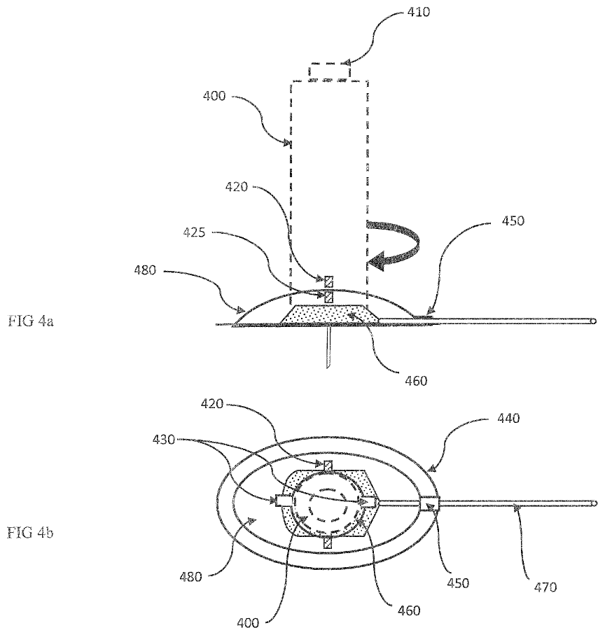

FIGS. 4a-4b shows an embodiment of the protective cover attached to an infusion set inserter for simultaneous application of the protective cover and infusion set.

The present disclosure provides a medical device accessory and methods related to embodiments of an infusion site protective cover or device that is designed to provide increased comfort to a patient as well as increased patient therapy compliance due to increased comfort.

The disclosed infusion site protective cover or device will lessen or prevent irritation and pain at the site by protecting it from the rub of clothing. It will also reduce movement of the cannula or needle due to applied pressure to the infusion site from sitting, leaning or laying on the site as this can pull the skin in various directions and cause movement of the infusion set hub. This can result in turning and pulling of the cannula or needle, as well as loosen the adhesive that is holding the infusion set in place. Once loosened moisture and contaminants may work their way in to the infusion site and cause irritation, pain, infections and swelling at the infusion site resulting in pain and discomfort to the patient.

FIGS. 1a-1d depict one example of an embodiment of a protective cover or device wherein the main body 100 has an outer surface raised convexly in an oblong geometric shape 160 and is further formed to have a flange 110 around the perimeter of the formed convex shape. The main body 100 may be formed of a rigid or semi rigid material such as paper/cardboard, wood, metal, plastic or glass. The flange has a geometric shape that facilitates providing a surface for adhering the protective cover to the patient's skin. The adhesive layer 120 is located on the flange, between the protective cover and the patient's skin, to provide adhesion of the protective cover to the patient's skin. The adhesive layer may or may not include padding to provide additional comfort to the patient. A groove or notch 130 in the flange accommodates the tubing that provides fluid connectivity from a medication delivery device to the infusion site cannula, needle or soft set, to pass freely under the protective cover so as to not become kinked or restrict fluid flow in any way. In the cutaway view point 170 the inner surface 140 is shown with the opening 150, on the underside of the protective cover, to provide clearance so the protective cover can be placed over the infusion set on the patient.

FIGS. 2a-2b depict another embodiment of the disclosed invention in a different geometric shape of a main body in the form of a round domed cover 200 with a flange 210 and a groove or notch 230 in the flange for tubing clearance as well as the adhesive layer 220. The protective cover is manufactured of a rigid or semi-rigid thermoplastic such as a for example a polyurethane, polypropylene, polyethylene, acrylic or other plastic material that can be cast, molded and thermo-formed in ways such as but not limited to injection molding or vacuum forming and several other methods that are obvious. FIG. 2a additionally depicts how the protective cover for an infusion site would be positioned on a patient with an infusion set installed. The infusion set consists of the infusion set hub 240 that connects and positions the cannula or infusion needle 260, as well as the tubing 250 providing fluid connectivity from the infusion pump to the infusion set, and the adhesive layer 225 that attaches and holds the infusion set to the patient's skin.

FIGS. 3a-3c show yet another embodiment wherein the protective cover main body is formed as a round donut shape 300 and has a hole through the center 310. This embodiment additionally has an adhesive layer 320 that is attached to the protective cover by the patient. While FIGS. 3a-3c depict a closed circle the protective cover may also be an open circle like that of a horse shoe or other geometric shape. The depicted protective cover is made of a soft polymer, rubber, foam or plastic gel material with a smooth outer surface and may be manufactured such that the inner cavity of the protective cover is solid or has many holes similar to a sponge or foam, i.e., open cell or closed cell foams, or the inner cavity maybe hollow in a continuous manner. There are many soft polymer or plastic materials that could be utilized for this purpose and are too numerous to mention. Material examples would include rubbers, epoxies, homopolymers, block polymers etc. specific examples such as polyacrylamides, polyethylenes, polypropylenes, polyethylene glycols and others including natural and synthetic materials that can form stable elastomers, gels and foams such as used extensively in industry. Methods to manufacture these materials are very well known to those that practice the art. Importantly the soft material needs to have a smooth somewhat slippery outside surface to allow clothing to glide across without catching and the material needs to deform and compress when pressed against and then spring back to its original shape to continue protecting the infusion site.

FIGS. 4a-4b depict another embodiment of the invention in which the protective device is removably attached to an infusion set inserter device 400 such that the protective cover 480 is attached or adhered to the patient simultaneously to the insertion of the infusion set 460 on the patient. These devices are commonly used to insert the infusion cannula or infusion needle into the subcutaneous region under the skin of the patient. The infusion set inserters are as varied as the different brands of infusion sets and are typically designed to fit one of each companies particular infusion set products. Most insertion devices are spring loaded and have a trigger or release button 410. This exemplary version shows a simple quarter turn boss and groove release mechanism. The bosses protrude outwardly from the insertion device. There is an upper boss 420 and a lower boss 425 that fit through matching grooves 430 in the hole at the top of the protective cover that the inserter fits through, by turning the infusion set inserter one quarter turn after the infusion set is inserted the inserter device is released. The patient can load the protective covers onto the insertion device for each use and only need to make sure that the infusion set tubing 470 is aligned with the groove or notch 450 in the flange 440 of the cover. There are many ways to reversibly attach the protective cover to an infusion set insertion device such that this is meant to demonstrate only one of many possible mechanical designs that could deliver the same or similar results of attaching the infusion set protector at the same time the infusion set is inserted.

Although a few specific embodiments have been described in detail above, other modifications consistent with the spirit of this disclosure are contemplated.

1. A protective cover for an infusion site, comprising:
a main body having an inner surface, an outer surface and a circumferential flange, wherein the body has a raised outer surface overlying a hollow cavity, and the flange has one or more grooves adapted to provide clearance for one or more infusion set tubes to pass between the patient's skin and the cover, when the protective cover is installed over an infusion site.

2. The protective cover of claim 1, further including adhesive or tape for fixing the cover to the patient's skin.

3. The protective cover of claim 1, wherein the main body is formed of a rigid or semi rigid material.

4. The protective cover of claim 3, wherein the rigid or semi rigid material is selected from the group consisting of paper, cardboard, wood, metal, plastic and glass.

5. The protective cover of claim 1, wherein the main body includes one or more holes for clearance and reversible attachment of an infusion set inserter.

6. The protective cover of claim 1, wherein the main body has one or more holes for air flow and access.

7. The protective cover of claim 1, wherein the cover has padding on the flange adapted to locate between the body and the patient's skin.

8. The protective cover of claim 1, wherein the main body raised outer surface has a rounded or elongated dome shape, or a round donut shape.

9. A protective cover for an incision site, comprising:
a main body having an upper surface, a bottom surface, and an exterior side surface defining a hollow; and,
a through hole adapted to accommodate encircling an infusion set, passing through the upper and bottom surfaces;
wherein the main body is formed of a resiliently a deformable plastic, gel or foam that returns to its original shape after being deformed.

10. The protective cover of claim 9, wherein the main body has a smooth exterior surface, and an adhesive on the bottom surface adapted to allow the protective cover to be adhered to the patient's skin.

11. The protective cover of claim 9, wherein the adhesive comprises a pressure sensitive adhesive, and having a release layer over the adhesive.

12. The protective cover of claim 9, wherein the main body has at least one vertical cut connecting the through hole to an outward exterior side surface of the main body.

13. A method of protecting an infusion site comprising providing a protective cover as claimed in claim 1, over the infusion site, and adhering the protective cover to patient's skin around the infusion site.

14. A method of protecting an infusion site comprising providing a protective cover as claimed in claim 9, over the infusion site, and adhering the protective cover to patient's skin around the infusion site.
